(19) United States
(12) Patent Application Publication
(10) Pub. No.: US 2009/0215889 A1
(43) Pub. Date: Aug. 27, 2009

(54) Novel Use of Zinc Gluconate for Treating Hydradenitis Suppurativa

(75) Inventors: Catherine Suck, Sceaux (FR); Brigitte Dreno, Nantes (FR); Jacques Bodin, Levallois-Perret (FR)

Correspondence Address: BUCHANAN, INGERSOLL & ROONEY PC, POST OFFICE BOX 1404, ALEXANDRIA, VA, 22313-1404, US

(73) Assignee: LABCATAL, Montrouge (FR)

(21) Appl. No.: 11/918,317

(22) PCT Filed: Apr. 6, 2006
(86) PCT No.: PCT/FR2006/000761

§ 371 Date: Apr. 13, 2009

(30) Foreign Application Priority Data
Apr. 13, 2005 (FR) 0503695

(51) Int. Cl. A61K 31/315

(52) U.S. Cl. 514/494

(57) ABSTRACT

The invention relates to a novel therapeutic use of zinc gluconate.

The zinc gluconate is used in a form for oral administration, at a daily dosage of between 45 and 125 mg, for treating suppurative hidradenitis, or Verneuil's disease.

The present invention relates to a novel therapeutic application of zinc gluconate, and more particularly a novel application of zinc gluconate administered orally for the treatment of suppurative hidradenitis, commonly known as Verneuil's disease, and also to a composition suitable for such a treatment.

### BACKGROUND OF THE INVENTION

Verneuil's disease, or suppurative hidradenitis, is an orphan disease, i.e. a relatively rare disease, for which few effective treatments have been proposed.

Verneuil's disease is a chronic affliction that develops by inflammatory and suppurative outbursts in anatomical areas comprising apocrine sweat glands. The anatomical structures that are affected are those of the pilosebaceous and sudoral unit (the apocrine sweat glands opening into the hair follicles). The initial lesion is probably an obstruction of the hair follicle, the apocrine sweat glands being secondarily affected. Clinically, the disease is characterized at the start by the periodic appearance of deep painful nodules without spontaneous opening on the skin, unlike acne and furuncles. Repetition of the outbursts of inflammatory nodules and their secondary opening on the skin lead to the formation of painful and malodorous suppurative fistulae, the scarring process forming lines that may result in the formation of vast suppurative and painful maculopapules.

The diagnosis is based on three factors: characteristic elementary lesions, a specific topography and the chronic or recurrent nature. The characteristic elementary lesions are painful or sensitive, closed hypodermic nodules, of over a centimeter in diameter, without spontaneous opening on the skin (at least at the start), abscesses, fistulae, infiltrated maculopapules, scar lines and polyporous comedones. The inflammatory lesions each have an average duration of 7 days, but the repetition of the outbursts and the coexistence of the factors have the effect that more than 50% of patients permanently have one or more painful nodules. The characteristic topographies are the armpits, the sub- and intermammary regions, and the inguino-crural and perianal regions. Less commonly, attack of the buttocks, the pubis, the chest and the retro-auricular areas is observed. The average duration of evolution of the disease is 19 years, the start frequently occurring around the age of puberty or in young adults and the disease having a tendency to disappear beyond the age of 50.

The severity is extremely varied, from a few nodules per year up to the permanent presence of an enormous suppurative maculopustule. A classification of this. severity was established by Hurley in 3 stages. This allows diseases to be classified between those that are probably cases of a stage I medical treatment and those that are definitely cases of a large stage III surgical excision. Complications of suppurative hidradenitis are rare, but some are serious, such as axial or peripheral inflammatory rheumatism. The overall frequency of cancers is increased in the case of these individuals: odd ratio (OR)=1.5; hepatic and oral cancers, but especially epithelial cancers, some occurring essentially in the genito-crural and anal regions with an odd ratio of 4.6.

Various studies have attempted to evaluate the prevalence, which is estimated at 1% of the overall population, and may rise up to 4% in young adult populations.

No treatment is entirely satisfactory, which explains the disarray of patients suffering from this often highly incapacitating affliction. Even in the “minor” forms, the repetition of the painful outbursts and of occasionally malodorous suppuration has a severe repercussion on the quality of life.

At the Hurley stage III and often stage II, the only course of action available is surgical excision of the entire affected area followed by covering, which is often done as a second-line treatment with or without a graft.

At stages I and I-II, various medical treatments have been attempted, with mixed results. Antiandrogens such as cyproterone acetate showed efficacy only at large doses (100 mg/day of cyproterone acetate) in the case of a small number of patients, with recurrence when the dose is reduced to 50 mg.

Radiotherapy has made it possible to obtain remission in the case of certain patients, without any observed side effect. However, given the specific spontaneous risk of cancer in the case of individuals suffering from suppurative hidradenitis, the use of radiotherapy involves a long-term potential risk that appears unreasonable.

Tests have recently been performed with hair-removing lasers, with encouraging results.

Oral isotretinoin gave very mediocre results in an open study on 80 patients. This result is not surprising, given the absence of sebaceous hypersecretion in this disease, unlike acne.

Antibiotics have been widely used, occasionally in the object of a disease of primarily infectious origin, this hypothesis nowadays being considered obsolete: the flora is usually polymicrobial, comprising staphylococci, streptococci, gram-negative bacilli and anaerobic organisms. Short-term treatments, either with anti-staphylococcus agents or with clindamycin, given at the start of an outburst in the hope of stopping it, give relatively mediocre results. Tests of long-term treatments with cyclins, on the model of what is done in the case of acne, with the aim of preventing the occurrence of outbursts, have not afforded satisfactory results.

There is thus at the present time no satisfactory medical treatment for attenuating or eliminating the inflammatory outbursts of the disease in its early stages.

It is known that certain zinc salts may be used therapeutically for treating certain dermatological complaints. More particularly, specialty products based on zinc gluconate are used orally for treating macrocystic or nodular inflammatory acne, or enteropathic acrodermatitis (see Vidal's Dictionary, published by OVP, Paris 2004). In the treatment of acne, zinc gluconate may also be combined with chitosan glycolate according to patent EP 981 325 and with a tannin-rich plant extract according to patent FR 2 765 108. It has also been proposed to use zinc gluconate for treating multiple sclerosis, for instance in patent EP 184 514 or for treating hyperprolactinemia as in patent EP 185 586. U.S. Pat. No. 5,208,031 describes the use of zinc gluconate, propionate and acetate for treating herpes. More recently, patent application US 2005/048 139 has proposed compositions combining zinc salts such as zinc gluconate, lactate and acetate for reducing the anti-irritant effect of antimicrobial compounds used in hygiene compositions.

Suppurative hidradenitis and acne are two different diseases. Hitherto, the treatments for acne have led, in the case of suppurative hidradenitis, to only mediocre and/or transient results.

The studies and experiments performed by the Applicant have revealed, unexpectedly, good anti-inflammatory efficacy for daily doses higher than the doses usually used in the treatment of acne, and have also made it possible to demonstrate that zinc gluconate can be used effectively in the treatment of Verneuil's disease.

### SUMMARY OF THE INVENTION

Thus, one subject of the present invention is the use of zinc gluconate in the preparation of a medicament for treating suppurative hidradenitis, and more particularly a medicament for oral administration.

A subject of the invention is also a novel composition based on zinc gluconate that may be used therapeutically, in a form for oral administration, intended for treating suppurative hidradenitis, and especially formulated for this treatment.

### DETAILED DESCRIPTION OF THE PREFERRED EMBODIMENTS

According to the present invention, the unit dose, i.e. the content of zinc gluconate in the composition, is generally between 15 and 90 mg and preferably between 15 and 45 mg per dosage intake.

The daily dosage is determined by the practitioner as a function of the patient's condition, but is generally between 45 and 125 mg.

The tolerance to these doses (for example 90 mg/day, i.e. 6 gel capsules containing a 15 mg dose) is usually good, the observed side effects are only digestive, and there is no systemic toxicity to be feared (the “Vidal” monograph, in the “overdose” section, is thus worded: “The probability of an acute intoxication is zero . . . ”).

The efficacy of zinc gluconate in the treatment of suppurative hidradenitis was verified by the clinical observation performed on 20 patients, 16 of whom were monitored for a period of up to 24 months.

20 patients, 14 women and 6 men, from 23 to 72 years old, whose history of the disease ranged from 6 to 13 years, were placed on 90 mg of zinc gluconate per day for a period of a few months to 3 years.

In 34% of the cases, the location of the lesions was inguinal, axillary in 24% of the cases, and on the buttocks in 17% of the cases. The other affected regions in 5% to 7% of the cases were the chest, the pubis or the perineum.

Under monitoring for several months, 9 of these patients (45%) showed a full remission of the disease, and 6 of them (30%) showed a partial remission. There are no data for 4 patients (treatment started too recently) and for one patient only, the treatment proved to be ineffective.

These data are reproduced in the table below:

| No. | Sex | Location | Dose 1 | Dose 2 | Rep | Monitoring |
|---|---|---|---|---|---|---|
| 1 | F | inguinal folds, | 90 mg | 60 mg | CR | 3 months |
| | | buttocks, armpits | | | | |
| 2 | M | ND | 75 mg | 75 mg | CR | >12 months |
| 3 | M | axillary, | 90 mg | 60 mg | PR | 8 months |
| | | presternal, | | | | |
| | | inguinal | | | | |
| 4 | M | scrotum, inguinal, | 90 mg | | PR | 7 months |
| | | buttocks | | | | |
| 5 | F | axillary, | 90 mg | 90 mg | PR | >12 months |
| | | perimammary | | | | |
| 6 | F | inguinal, supra- | 90 mg | | PR | 6 months |
| | | pubic, breasts, | | | | |
| | | inter-buttock fold | | | | |
| 7 | M | axillary, pelvic | 90 mg | 30 mg | CR | >12 months |
| 8 | F | axillary, inguinal | 90 mg | | | |
| 9 | F | axillary | 90 mg | 15 mg | CR | >12 months |
| 10 | F | inguinal, inter- | 120 mg | ND | PR | ND |
| | | buttock, peri- | | | | |
| | | vulvar | | | | |
| 11 | F | axillary, inguinal | 90 mg | ineff. | | |
| 12 | F | inguinal, | 90 mg | 60 mg | CR | 4 months |
| | | buttocks, axillary | | | | |
| 13 | F | axillary, inguinal | 90 mg | | | |
| 14 | M | inguinal, | 90 mg | 15 mg | CR | 4 months |
| | | buttocks, abdomen | | | | |
| 15 | M | axillary, inguinal | 120 mg | | PR | 5 months |
| 16 | F | ND | 60 mg | | | |
| 17 | F | inguinal, | 90 mg | 30 mg | CR | >12 months |
| | | axillary, buttocks | | | | |
| 18 | F | inguinal | 60 mg | | CR | 7 months |
| 19 | F | inguinal | 90 mg | 30 mg | CR | >12 months |
| 20 | F | inguinal | 90 mg | ND | | ND |

CR: complete remission
PR: partial remission
ND: not documented

“Dose 1” is the dose administered at the start of the treatment. “Dose 2” is the maintenance dose. The medical monitoring was generally from 4 to 8 months, and in a few cases it was extended beyond 12 months.

These results show that zinc gluconate shows satisfactory efficacy in the treatment of suppurative hidradenitis when it is administered orally at relatively high doses, i.e. of the order of 60 to 120 mg per day for the attack treatment, and 15 to 90 mg per day for the maintenance treatment.

The composition according to the invention may be in the form of gel capsules, wafer capsules, tablets, a drinkable solution, effervescent tablets or any other usual form for oral administration.

Besides zinc gluconate, the composition may contain suitable excipients as a function of the intended use, such as diluents, for example lactose or mannitol, lubricants, for example magnesium stearate or hydrated colloidal silica, and a disintegrant, for example wheat starch or pregelatinized starch.

Other excipients may be used as a function of the intended mode of administration.

Exemplary embodiments, which are in no way limiting, are given below to illustrate the invention.

Formulation 1: gel capsule
A gel capsule having the composition below
is prepared via the usual techniques:

| | | |
|---|---|---|
| Zinc gluconate (expressed as zinc metal) | 15.0 | mg |
| Magnesium stearate | 6.0 | mg |
| Levilite | 6.0 | mg |
| Tixosil | 2.0 | mg |
| Wheat starch | 27.7 | mg |
| Lactose | qs 200.0 | mg |

These amounts are indicated for a size 2 gel capsule.

Formulation 2: tablet
Using the usual techniques, a tablet having
the composition below is prepared:

| | | |
|---|---|---|
| Zinc gluconate (expressed as zinc metal) | 15.0 | mg |
| PEG 6000 | 5.76 | mg |
| Primojel | 7.2 | mg |
| Compritol 888 | 5.76 | mg |
| Sorbitol | qs 210.0 | mg |

Formulation 3: film-coated tablet
Using the usual techniques, a tablet having
the composition below is prepared:

| | | |
|---|---|---|
| Zinc gluconate (expressed as zinc metal) | 15.0 | mg |
| PEG 6000 | 5.76 | mg |
| Primojel | 7.2 | mg |
| Compritol 888 | 5.76 | mg |
| Sepisperse | 8.2 | mg |
| HP50 | 10.81 | mg |
| Dibutyl phthalate | 1.09 | mg |
| Sorbitol | qs 210.0 | mg |

Formulation 4: effervescent tablet
An effervescent tablet having the composition
below is prepared via the usual techniques:

| | | |
|---|---|---|
| Zinc gluconate (expressed as zinc metal) | 30.0 | mg |
| Anhydrous citric acid | 0.96 | g |
| PVP K90 | 1.0 | mg |
| Sodium saccharin | 5.0 | mg |
| Sodium bicarbonate | 1.194 | g |
| Sodium carbonate | 60.0 | mg |
| L-Leucine | 5.76 | mg |
| Sepisperse | 125.0 | mg |
| HP50 | 10.81 | mg |
| Dibutyl phthalate | 1.09 | mg |

| | |
|---|---|
| Mannitol | qs one tablet |
| | weighing 2.657 g |

Formulation 5: gel capsule
A gel capsule having the composition below
is prepared via the usual techniques:

| | | |
|---|---|---|
| Zinc gluconate (expressed as zinc metal) | 45.0 | mg |
| Magnesium stearate | 6.0 | mg |
| Levilite | 6.0 | mg |
| Tixosil | 2.0 | mg |
| Wheat starch | 27.7 | mg |
| Lactose | qs 300.0 | mg |

These amounts are indicated for a size 1 gel capsule.

Formulation 6: sachet of effervescent powder
A sachet of effervescent powder having the composition
below is prepared via the usual techniques:

| | | |
|---|---|---|
| Zinc gluconate (expressed as zinc metal) | 30.0 | mg |
| Anhydrous citric acid | 1.086 | g |
| Aspartame | 20.0 | mg |
| Sodium bicarbonate | 1.316 | g |
| Sodium carbonate | 60.0 | mg |
| L-Leucine | 75.0 | mg |

| | |
|---|---|
| Mannitol | qs one sachet |
| | containing 3.0 g |

1. (canceled)

2. The method as claimed in claim 6, wherein the zinc gluconate is administered orally.

3. The method as claimed in claim 2, wherein the zinc gluconate is administered in the form of a tablet, an effervescent tablet, a gel capsule, a wafer capsule, a drinkable solution or a powder.

4. The method as claimed in claim 3, wherein the zinc gluconate is administered in unit doses of between 15 and 90 mg.

5. The method as claimed in claim 6, wherein the daily dosage of the zinc gluconate is between 45 and 125 mg.

6. A method of treating suppurative hidradenitis comprising administering a therapeutically effective amount of zinc gluconate.

7. An oral pharmaceutical composition comprising a therapeutically effective amount of zinc gluconate and pharmaceutically acceptable excipients.

8. The oral pharmaceutical composition of claim 7, wherein the zinc gluconate is in an amount of between 15 and 90 mg.

9. The oral pharmaceutical composition of claim 8, wherein the zinc gluconate is in an amount of between 15 and 45 mg.

10. The oral pharmaceutical composition of claim 7, wherein the composition in the form of a tablet, an effervescent tablet, a gel capsule, a wafer capsule, a drinkable solution or a powder.

11. The oral pharmaceutical composition of claim 7, wherein the pharmaceutically acceptable excipients are selected from the group consisting of lactose, mannitol, magnesium stearate, hydrated collodial silical, wheat starch, pregelatinized starch, and mixtures thereof.
